(19) United States
(12) Patent Application Publication
(10) Pub. No.: US 2009/0198310 A1
(43) Pub. Date: Aug. 6, 2009

(54) BODY WARM-KEEPING APPARATUS FOR KEEPING THE CORE TEMPERATURE OF A HUMAN BODY DURING AN X-RAY EXAMINATION

(75) Inventor: Yu-Mei CHEN, Taichung County (TW)

Correspondence Address: BROWDY AND NEIMARK, P.L.L.C.;624 NINTH STREET, NW, SUITE 300, WASHINGTON, DC, 20001-5303, US

(73) Assignee: CHARDER ELECTRONIC CO., LTD., Dali City (TW)

(21) Appl. No.: 12/102,142

(22) Filed: Apr. 14, 2008

(30) Foreign Application Priority Data
Feb. 5, 2008 (TW) 97202536

(51) Int. Cl. A61F 7/00

(52) U.S. Cl. 607/108; 607/96

(57) ABSTRACT

A body warm-keeping apparatus for keeping the core temperature of a human body during an X-ray examination is disclosed to include an apparatus body providing a platform for supporting an object to receive an X-ray examination, a heater device or a number of heater devices that allow penetration of X-rays therethrough and are arranged at the platform for the contact of the object to keep the object warm, one or a number of accommodation compartments disposed at one side relative to the platform, said platform defining a radiation region corresponding to an X-ray radiation path, said at least one accommodation compartment being disposed in said X-ray radiation path, said at least one accommodation compartment being adapted for accommodating a photographic receptor adapted for producing a shadow image of the internal structure of a part of the object being X-rayed. The space-saving arrangement of the heater device(s) facilitates design and fabrication of the body warm-keeping apparatus.

### BACKGROUND OF THE INVENTION

1. Field of the Invention

The present invention relates to warm-keeping apparatus and more specifically, to a body warn-keeping apparatus for keeping the core temperature of an object during an X-ray examination.

2. Description of the Related Art

When making an X-ray examination, the core temperature of the object to be X-rayed may have to be kept. For example, when a premature or weak baby is going to receive an X-ray examination, a medical person may cover a blanket on the patient or put a blanket on the platform beneath the patient to kept the body of the patient warm, avoiding the patient losing temperature that may risk the patient's life.

However, a blanket does not provide excellent warm-keeping effect, and is difficult to wash. After one use, the blanket may be discarded. Therefore, using a blanket to keep a patient warm during an X-ray examination is not an economic way. Further, a premature or weak baby or a temperature-losing patient shall have to be kept in an infant incubator/medical bed that keeps the core temperature of the baby/temperature-losing patient stable.

When a temperature-losing patient is going to receive an X-ray examination, the patient must be delivered from the medical bed to the X-ray system. It is difficult to move the patient from the medical bed to the X-ray system. This procedure takes much time and labor, and causes inconvenience to medical persons. To a temperature-losing patient carrying a life-support system, the problem will get worse, and the X-ray examination may be unable to perform on time, wasting precious time.

Further, when a premature baby is going to receive an X-ray examination, the medical person needs to carry the premature baby from the infant incubator to the platform of the X-ray machine. After the examination, the medical person needs to carry the premature baby from the platform of the X-ray machine back to the infant incubator. This procedure wastes much time and labor, and interferes with the baby's sleep quality. To a baby carrying a life-support system, this procedure not only wastes time and labor but also increases life-threatening risk. Further, the warm-keeping effect of wrapping a premature baby with a blanket is limited.

To avoid the aforesaid problems, a warm-keeping apparatus is known comprising a platform for the placing of a blanket, an X-ray tray provided beneath the platform for holding an X-ray film, and a hot air apparatus adapted for providing hot air to keep the patient warm. This warm-keeping apparatus allows the patient therein to receive an X-ray examination directly without interference, facilitating performance of an X-ray examination rapidly and safely.

To avoid interference with the radiation of X-rays, the arrangement of the hot air apparatus and other devices (life-support device and etc.) is complicated. The arrangement includes an opening in the X-ray radiation path and other complicated structures to avoid blocking X-rays and affecting examination accuracy. Further, the cost of the hot air apparatus is high. The hot air apparatus consumes much energy and causes noises during its operation.

Therefore, it is desirable to provide a body warm-keeping apparatus that eliminates the aforesaid drawbacks and problems.

### SUMMARY OF THE INVENTION

The present invention has been accomplished under the circumstances in view. It is the main object of the present invention to provide a body warm-keeping apparatus, which provides excellent body warm-keeping effect and facilitates the performance of X-ray examinations. It is another object of the present invention to provide a body warm-keeping apparatus, which has the parts thereof skillfully arranged, effectively lowering the cost.

To achieve these and other objects of the present invention, the body warm-keeping apparatus is adapted for keeping the core temperature of a human body during an X-ray examination, comprising an apparatus body and at least one heater device. The apparatus body comprises a platform and at least one accommodation compartment disposed at one side relative to the platform. The platform defines a radiation region corresponding to an X-ray radiation path. The at least one accommodation compartment is disposed in the X-ray radiation path, for accommodating a photographic receptor. The at least one heater device is mounted on the radiation region of the platform and kept in contact with the human body receiving an X-ray examination. Each heater device comprises a conducting material and a conducting wire. The conducting material is arranged corresponding to the radiation region. The conducting wire electrically connects a power supply unit to the conducting material, and extends along the border of the conducting material. The conducting material allows penetration of X-rays therethrough for causing the photographic receptor to produce a shadow image of the internal structure of a part of the human body that is kept in contact with the at least one heater device and radiated with X-rays going through the X-ray radiation path.

In conclusion, the heater device of the body warm-keeping apparatus allows penetration of X-rays therethrough, and is arranged on the platform and kept in direct contact with the patient. The layout of the internal conducting wire of the heater device avoids interfering with the patient during an X-ray examination. In addition to excellent warm-keeping effect, the body warm-keeping apparatus of the present invention allows the patient to receive an X-ray examination directly, facilitating medical performance. Further, the design arrangement of the invention is easy, having an industrial value and market competitive power.

### BRIEF DESCRIPTION OF THE DRAWING

FIG. 1 is a perspective view of a body warn-keeping apparatus in accordance with a first embodiment of the present invention.

FIG. 2 is a schematic drawing showing the structure of the heater device used in the body warm-keeping apparatus in accordance with the first embodiment of the present invention.

FIG. 3 is a sectional view in an enlarged scale of a part of FIG. 2.

FIG. 4 illustrates an image formed on the photographic receptor subject to an application of the body warm-keeping apparatus in accordance with the first embodiment of the present invention.

FIG. 5 illustrates the positioning relationship between the radiation region and another design of heater device according to the present invention.

FIG. 6 is similar to FIG. 4, illustrating the image of the conducting wire excluded from the photographic receptor.

FIG. 7 is a schematic applied view of a body warm-keeping apparatus in accordance with a second embodiment of the present invention.

FIG. 8 is a schematic applied view of a body warm-keeping apparatus in accordance with a third embodiment of the present invention.

### DETAILED DESCRIPTION OF THE INVENTION

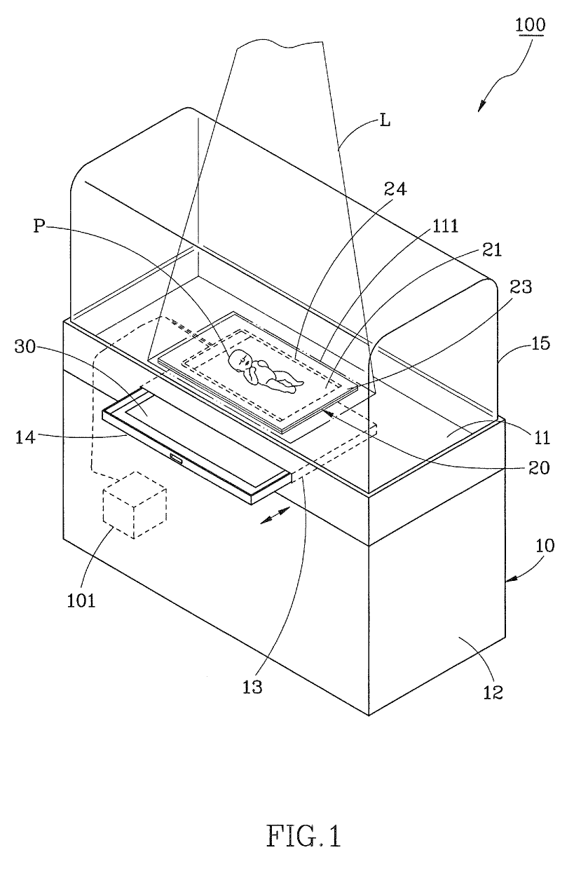

Referring to FIG. 1, a body warm-keeping apparatus 100 for use during an X-ray photography application to keep the core temperature of a human body to be photographed in accordance with a first embodiment of the present invention is made in the form of an infant incubator. However, the body warm-keeping apparatus is not limited to the form of an infant incubator. According to this first embodiment, the body warm-keeping apparatus 100 is comprised of an apparatus body 10 and a heater device 20, and adapted for keeping the core temperature of an object P to be photographed (for example, a premature or weak baby).

The apparatus body 10 can be constructed subject to the architecture of a commercial infant incubator, comprising a platform 11 on which the object (premature or weak infant) P is lying, a base 12 joined to the bottom side of the platform 11, and a hood 15 movable relative to the platform 11 between an open position for complete access to the object P and a close position to enclose the platform 11.

The platform 11 has a radiation region 111 corresponding to an X-ray radiation path L for the mounting of the heater device 20.

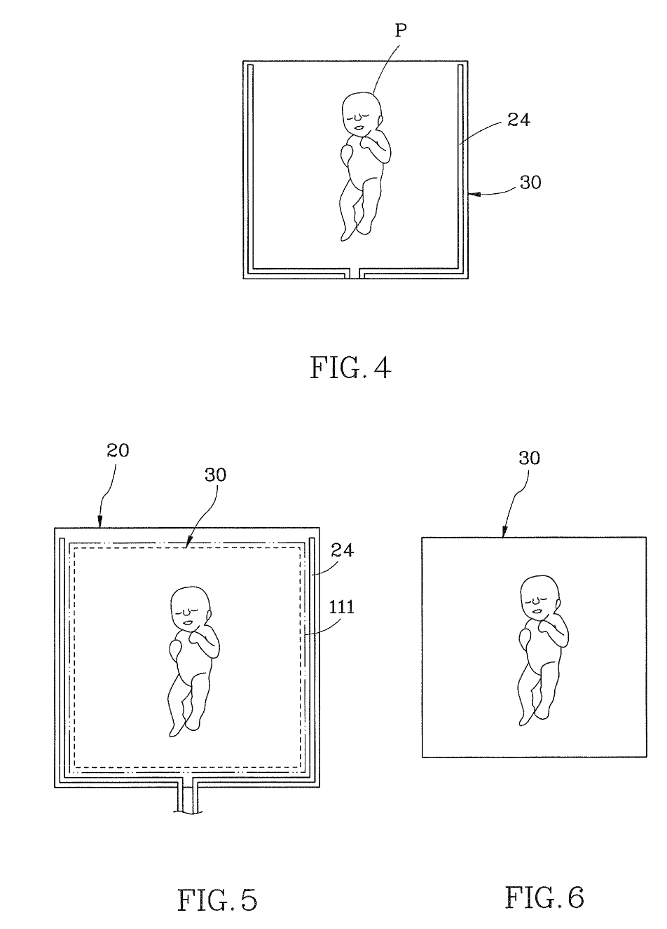

The base 12 comprises an accommodation compartment 13 corresponding to the radiation region 111, a tray 14 that can be inserted into or pulled out of the accommodation compartment 13 by an operator so that the operator can put a photographic receptor (for example, photographic film) 30 into the tray 14 for photography or take the photographic receptor 30 out of the tray 14 after photographed. According to the this embodiment, the photographic receptor 30 can be any means capable of receiving the radiation of X-ray and forming thereon the image of the object P (see FIG. 4). The base 12 of the apparatus body 10 has installed therein a power supply unit 101 (for example, AC/DC converter) for providing the body warm-keeping apparatus 100 with the necessary electric power for operation. Further, a controller (not shown) is provided for controlling the power output of the power supply unit 101 to regulate the amount of thermal energy produced by the heater device 20.

Referring to FIGS. 2 and 3 and FIG. 1 again, the heater device 20 is mounted on the radiation region 111 for direct contact with the object P (the direct contact can be achieved by means of covering the heater device 20 on the object P for direct contact, or putting the heater device 20 under the object P). According to this embodiment, the heater device 20 is a flexible heat-emitting member that allows penetration of X-rays therethrough. The heater device 20 is comprised of a thin-sheet conducting material 21, a flexible base material 22, a warm-keeping bag 23, and a conducting wire 24.

The thin-sheet conducting material 21 is a flexible electroheating material that allows penetration of X-rays therethrough. According to this embodiment, the thin-sheet conducting material 21 is prepared from, but not limited to, carbon fiber. Further, the thin-sheet conducting material 21 can be made in, but not limited to, the form of a thin piece of cloth, thin sheet of felt or thin film.

The flexible base material 22 is formed of two flexible thin films that surround the thin-sheet conducting material 21 and allows penetration of X-rays therethrough. The flexible base material 22 supports and protects the thin-sheet conducting material 21.

The warm-keeping bag 23 can be made to detachably wrap the flexible base material 22. Alternatively, the warm-keeping bag 23 can be fixedly fastened to the flexible base material 22. According to this embodiment, the warm-keeping bag 23 is made of a flexible thermal insulation material such as silicon rubber or plastic foam material, and therefore the warm-keeping bag 23 effectively lowers the heat dissipation speed of the thin-sheet conducting material 21, extending the warm-keeping time and saving the energy. However, the warm-keeping bag 23 is not a requisite element and can be omitted. Further, the warm-keeping bag 23 may be wrapped with a layer of waterproof covering material (not shown) to enhance water resistance. For example, the warm-keeping bag 23 can be covered with a layer of waterproof nylon fabric covering to match with the thin-sheet conducting material 21 and the flexible base material 22, so that the heater device 20 imparts comfort to the user and can be washed and kneaded in water without losing its function.

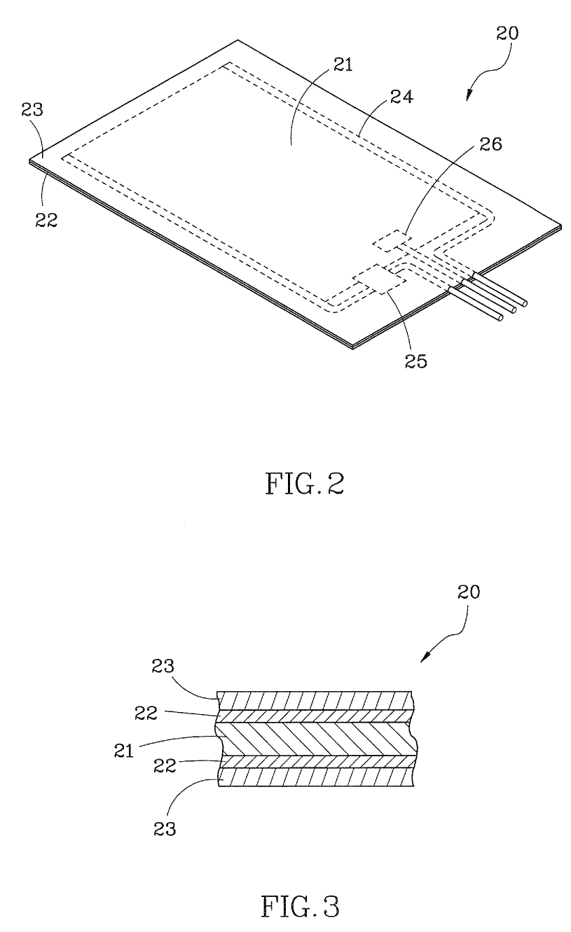

The conducting wire 24 is electrically connected with the thin-sheet conducting material 21 and the power supply unit 101 of the apparatus body 10 to transmits output electric energy from the power supply unit 101 to the thin-sheet conducting material 21, causing the thin-sheet conducting material 21 to produce heat. According to this embodiment, as shown in FIGS. 1 and 2, the conducting wire 24 extends along the border of the thin-sheet conducting material 21 to avoid blocking the X-ray when the object P is being photographed, so as not to affect the image forming result, i.e., the arrangement of the conducting wire 24 must not block the X-ray from passing through the object P.

The material for the hood 15 allows penetration of X-rays therethrough. According to this embodiment, the hood 15 can be made having ventilation holes or equipped with an air circulation system (not shown) to maintain ventilation. This part is not within the scope of the present invention, no further detailed description in this regard is necessary.

In conclusion, when compared with the use of a blanket according to the prior art, the heater device 20 of the present invention has better warm-keeping effect and is relatively easier to clean. When compared with the use of a hot air equipment according to the prior art, the heater device 20 of the present invention has the advantages of small size, less space occupation, ease of arrangement Furthermore, the heater device 20 allows penetration of X-rays therethrough so that a medical person can directly apply an X-ray examination to the object P without moving the object P from the body warm-keeping apparatus 100, saving much labor and time. Further, the layout of the conducting wire 24 of the heater device 20 is kept away from the area bearing the object P, and therefore the heater device 20 does not interfere with the performance of an X-ray examination that is being applied to the object P. When compared with the arrangement of the conventional hot air equipment, the design and preparation cost of the present invention is relatively cheap, and the body warm-keeping apparatus 100 provides extra space for accommodating other devices to provide added value.

After understanding of the arrangement and relative positions of the component parts of the body warm-keeping apparatus 100, the operation and effects of the body warm-keeping apparatus 100 are outlined hereinafter.

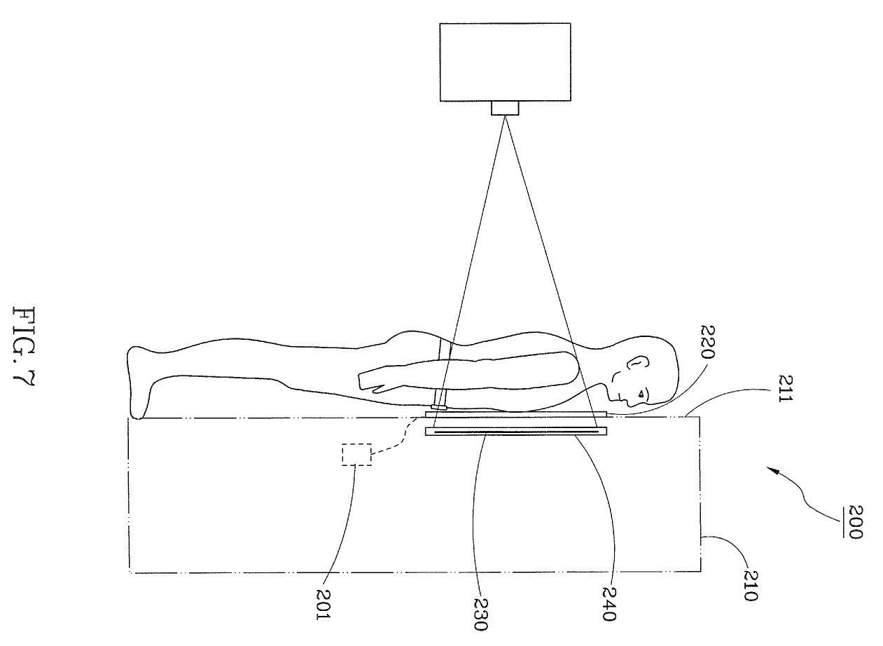

When applying an X-ray examination to the object P in the body warm-keeping apparatus 100, the X-ray source is started to emit X-rays through the hood 15, the object P, the heater device 20 and the platform 11, and the radiation is then received by the photographic receptor 30 to produce an X-ray image after exposure, as shown in FIG. 4. The image includes the object P and the conducting wire 24 around the border of the heater device 20. The layout of the conducting wire 24 does not interfere with the object P when the object P is receiving an X-ray examination. Further, preventing presence of the conducting wire 24 in the image obtained from the photographic receptor 30 can be achieved by means of increasing the surface area of the heater device 20 to have the conducting wire 24 be arranged outside the radiation region 110, as shown in FIG. 5. FIG. 6 illustrates an image obtained from the photographic receptor 30 after an X-ray examination where the conducting wire 24 is not shown. The image shown in FIG. 6 facilitates reading. Of course, by means of contracting the surface area of the photographic receptor 30 can also achieve the same effect.

As stated above, the object P is kept in contact with the heater device 20 during an X-ray examination, therefore an excellent warn-keeping effect is obtained. Further, because the heater device 20 has light and thin characteristics and allows penetration of X-rays therethrough, as well as the advantage of the special layout of the conducting wire 24, the arrangement of the heater device 20 in the body warm-keeping apparatus 100 is easy. Therefore, the cost of the body warn-keeping apparatus 100 can be relatively reduced while image quality is maintained. The body warm-keeping apparatus 100 allows a medical person to conveniently apply an X-ray examination to the object P carried therein. Further, the conducting material 21 consumes low power, and therefore the heater device 20 is durable and safe in use, and the matched power supply unit 101 can be a small-sized design. Further, because the body warm-keeping apparatus 100 does not use any pump to provide hot air, the operation of the body warm-keeping apparatus 100 is smooth and making no noise, not interfering with sleep or rest of the object (infant) P.

Any suitable material that allows penetration of X-rays may be provided between the heater device 20 and the object (premature or weak baby) P, preventing from keeping the object (premature or weak baby) P too warm. Further, as shown in FIG. 2, an overheat protection switch (for example, bimetal switch) 25 may be installed in the conducting wire 24 of the heater device 20 for overheat protection. A temperature sensor 26 may be installed in the heater device 20 to detect the temperature of the conducting material 21. Through the temperature sensor 26, the user knows the current temperature of the conducting material 21. When the temperature surpassed a predetermined value, the overheat protection switch 25 turns off the circuit of the conducting wire 24 to stop the supply of electricity from the power supply unit 101 to the heater device 20, avoiding danger to the object P when the temperature sensor 26 malfunctions and assuring high safety of the body warm-keeping apparatus 100.

Further, the aforesaid accommodation compartment 13, heater device 20, photographic receptor 30 and radiation region 111 are not limited to one single number. For example, to satisfy the demand for photographing many parts of a premature or weak baby by X-rays at a time, the body warm-keeping apparatus can be made having multiple accommodation compartments for accommodating a respective tray for photographic receptor (photographic film) corresponding to multiple heater devices in multiple radiation regions on the platform of the apparatus body. Alternatively, the body warm-keeping apparatus can be made having one single heater device that can be moved in the platform to one of a number of locations for enabling one of a number of parts of the body of the object being carried on the heater device to be photographed by X-rays. A big area heater device may be used to substitute for a number of relatively smaller heater devices in matching with the multiple accommodation compartments design. Further, the photographic receptor 30 may be directly put in the accommodation compartment 13 without the aforesaid tray 14.

Further, the power supply unit 101 can be a DC battery directly installed in the apparatus body 10, or a discrete DC battery disposed outside the apparatus body 10. DC battery allows the medical person to use the body warm-keeping apparatus 100 conveniently, extending the moving distance and application range of the body warm-keeping apparatus 100. Alternatively, the power supply unit 101 can be a device that outputs AC power supply.

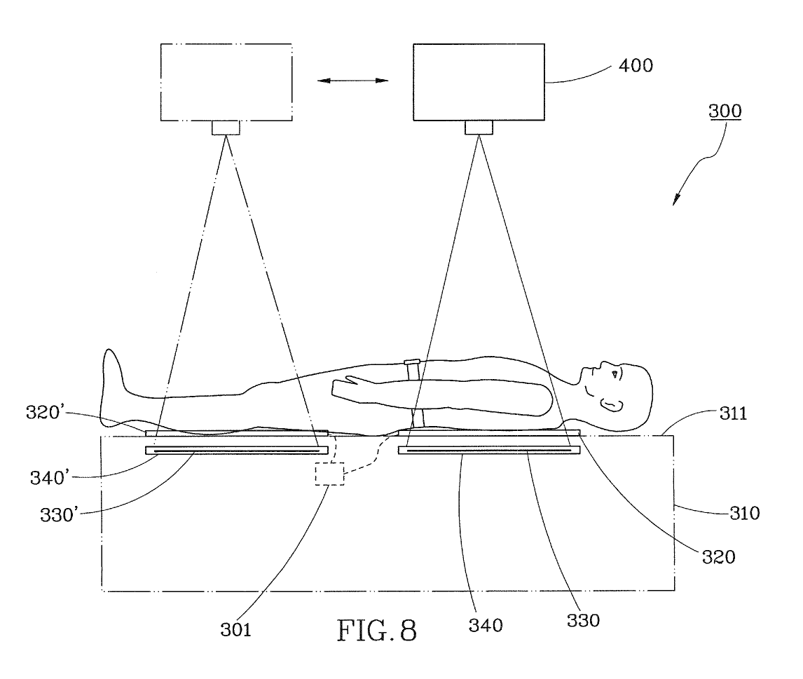

FIG. 7 illustrates an application of a body warm-keeping apparatus 200 in accordance with a second embodiment of the present invention. The design of this second embodiment is for supporting the object in a standing position for receiving an X-ray examination. FIG. 8 illustrates an application of a body warm-keeping apparatus 300 in accordance with a third embodiment of the present invention. The design of this third embodiment is for supporting the object in a lying position for receiving an X-ray examination. Same as the aforesaid first embodiment of the present invention, the body warm-keeping apparatus 200 of the second embodiment and the body warm-keeping apparatus 300 of the third embodiment commonly comprise an apparatus body 210 or 310, a heater device 220 or 320, a photographic receptor 230 or 330, and an accommodation compartment 240 or 340. The second and third embodiments of the present invention are designed for an adult user. The heater device 220 or 320 is directly bonded to the platform 211 or 311 or fixed thereto by means of any suitable fixation measures. The power supply unit 201 or 301 in the apparatus body 210 or 310 provides the heater device 220 or 320 with the necessary electric energy for working. The heater device 220 or 320 can be packaged with a DC battery, and then attached to the apparatus body 210 or 310 before an X-ray examination. The body warm-keeping apparatus 300 of the third embodiment shown in FIG. 8 further comprises a second heater device 320′, a second photographic receptor 330′, and a second accommodation compartment 340′. The two heater devices 320 and 320′ use one common power supply unit 301. The medical person can move the X-ray source 400 to photograph the chest and legs of the patient by X-rays. Alternatively, the body warm-keeping apparatus can be equipped with one single heater device that can be shifted between two locations corresponding to different photographic receptors for photographing two different parts of the patient. Further, one single big-size heater device (about the same size of the platform 311) may be used to substitute for the arrangement of the two heater devices 320 and 320′ for full body X-ray photograph.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A body warm-keeping apparatus for keeping the core temperature of a human body during an X-ray examination, the body warm-keeping apparatus comprising:
an apparatus body, said apparatus body comprising a platform and at least one accommodation compartment disposed at one side relative to said platform, said platform defining a radiation region corresponding to an X-ray radiation path, said at least one accommodation compartment being disposed in said X-ray radiation path, said at least one accommodation compartment being adapted for accommodating a photographic receptor; and
at least one heater device mounted on said radiation region of said platform and kept in contact with the human body receiving an X-ray examination, each said heater device comprising a conducting material and a conducting wire, said conducting material being arranged corresponding to said radiation region, said conducting wire electrically connecting a power supply unit to said conducting material and extending along the border of said conducting material, said conducting material allowing penetration of X-rays therethrough for causing said photographic receptor to produce a shadow image of the internal structure of a part of the human body being kept in contact with said at least one heater device and radiated with X-rays going through said X-ray radiation path.

2. The body warm-keeping apparatus as claimed in claim 1, wherein said apparatus body comprises at least one tray movably mounted on said at least one accommodation compartment for holding said photographic receptor.

3. The body warm-keeping apparatus as claimed in claim 1, wherein said conducting wire is arranged outside said radiation region.

4. The body warm-keeping apparatus as claimed in claim 1, wherein said heater device comprises an overheat protection switch installed in said conducting wire.

5. The body warm-keeping apparatus as claimed in claim 4, wherein said heater device comprises a temperature sensor adapted for detecting the temperature of said conducting material.

6. The body warm-keeping apparatus as claimed in claim 1, wherein said power supply unit outputs DC power supply.

7. The body warm-keeping apparatus as claimed in claim 1, wherein said power supply unit outputs AC power supply.

8. The body warm-keeping apparatus as claimed in claim 1, wherein said conducting material is a thin sheet member of carbon fiber.

9. The body warm-keeping apparatus as claimed in claim 1, wherein said heater device comprises a flexible base material enclosing said conducting material.

10. The body warm-keeping apparatus as claimed in claim 9, wherein said heater device comprises a warm-keeping bag wrapping said flexible base material.
